(19) United States
(12) Patent Application Publication
(10) Pub. No.: US 2020/0293926 A1
(43) Pub. Date: Sep. 17, 2020

(54) Infinite Edge-Computing Oracle

(71) Applicant: Anuthep Benja-Athon, New York, NY (US)

(72) Inventor: Anuthep Benja-Athon, New York, NY (US)

(73) Assignee: Anuthep Benja-Athon, New York, NY (US)

(21) Appl. No.: 16/351,540

(22) Filed: Mar. 13, 2019

(51) Int. Cl. G06N 5/04; G16H 50/20; H04L 29/06

(52) U.S. Cl. CPC G06N 5/043; H04L 67/104; H04L 63/0853; G16H 50/20

(57) ABSTRACT

The edge-computing and edge-exchanging individual and individual groups of mobile*_n-to-mobile*_n communication devices for rendering the Infinite edge-computing and edge-exchanging Oracle possessing abiotic thinking and reasoning for rendering definitive and specific continuum*_n of vital goods vital goods, products, services and resources in most industries.

This is a continuation-in-part of Ser. No. 14/606,956 entitled Billion Recounts of Events Ahead of Days filed Jan. 27, 2015, Ser. No. 14/721,981 entitled Global Healthcare Exchange filed May 26, 2015, Ser. No. 14/992,773 entitled the Creation of Abiotic-Biotic Civilization filed Jan. 11, 2016, Ser. No. 15/292,080 entitled EM Oracle filed Oct. 12, 2016, Ser. No. 15/495,164 entitled AIR Oracle filed Apr. 24, 2017, Ser. No. 15/727,644 entitled the Preemptions of Chronic Diseases filed Oct. 9, 2017, Ser. No. 15/881,805 entitled Abiotic Intelligence-Rendered Pay filed Jan. 29, 2018 and Ser. No. 16/166,133 entitled the Abiotic elligence-Rendered Thinking Faculty filed Oct. 21, 2018.

### FIELD OF THE INVENTION

Edge-computing and edge-exchanging mobile*_n-to-mobile*_n devices

### BACKGROUND OF THE INVENTION

In the age of networking communication devices via wireless, Wi-Fi and cable means, the innovative and unique infinite edge-computing and edge-exchanging Oracle is the omnipotent and omnipresent technology heretofore superseding all online technologies to rapidly and forever change and evolve the status quo information, inteligence, daa and services businesses and commerce in all industries thence evolving the present-future civilizations on earth and in space for milleniums to come. In other words, in this universe, Infinite edge-computing and edge-exchanging Oracle 24/7 AIR supplanting brains' infinite thoughts and guesses effecting random processes, events and circumstances with poor outcomes and adverse consequences including morbidity and mortality.

The Infinite edge-computing and edge-exchanging Oracle (known herein as ECO*_n) is the omnipotent and omnipresent infinite edge-computing and edge-exchanging technology implemented as app, apps or otherwise in, between and among billions of status quo networked mobile*_n-to-mobile*_n communication devices—whose users must-have and must-use said infinite edge-computing and edge-exchanging technology for all vital reasons for the best life and longevity—such as, but not limited to, billions of smartphones and laptops which are located albeit constantly and virtually mobile due to cable, satellite and wireless connectivities between and among place*_n on earth, other planet*_n and outer space so that said mobile*_n-to-mobile*_n communication devices whose relational data including demographics and relational data including continuum*_n of diagnoses of acute, subacute and chronic diseases and disorders and sequelae and related or relevant vital goods, products, services and resources in diverse industries and relations of, between and among said continuum*_n, unique IP*_n and ID*_n of said devices and adaptable security token*_n (henceforth collectively known as adaptable device*_n digital footprint*_n, between and among storage means in, between and among said devices and stationary devices and servers)are in and constantly and virtually being and existed and remained at any time*_n in, between and among same, different and diverse individual adaptable edge-computing and edge-exchanging Cell*_n being located and mobile in, between and among place*_n on earth, other planet*_n and outer space (known herein as EMC*_n).

In other words, ECO*_n comprising lattices of multi-dimensional relational databases comprising integral snippets*_n and relations of integral snippets*_n in said networked repositories of said networked mobile*_n-to-mobile*_n communication devices wherein ECO*_n for rendering and successively editing and updating improving and expanding individual networked mobile*_n-to-mobile*_n repositories and relations of, between and among said individual networked mobile*_n-to-mobile*_n repositories in individual group*_n of mobile*_n-to-mobile*_n repositories comprising same, similar and/or equivalent device*_n digital footprint*_n (known herein as footprint*_n) being same, similar and/or equivalent integral snippets*_n and relations of same, similar and/or equivalent relational data in, between and among footprint*_n in, between and among continuum*_n in said lattices in said storage means of, between and among said billions (henceforth collectively known as adaptable individual*_n and adaptable individual group*_n aka individual*_n and individual group*_n) wherein individual*_n and individual group*_n rendering and possessing inherent constantly shifting, expanding, retracting and adaptable ubiquitously networking mobile domains in, between, among and over place*_n on earth, other planet*_n and outer space in EMC*_n.

From every technological perspective and ground and space logistics, the crucial innovations are the innovative computer programs and software for rendering and successively adapting, improving and expanding ECO*_n for supplanting said status quo by rendering and successively adapting, improving and expanding individual*_n and individual group*_n. in EMC*_n thence rendering and successively adapting, improving and expanding ECODI*_n thence successively adapting, improving and expanding ECO*_n thence successively adapting, improving and expanding individual*_n and individual group*_n to harnessing and using immense processing powers of billions of processor units of mobile phones and laptops and individual group*_n of said processor units attached to and associated with storage means of said billions storage means of billions of terabits—immensely more superior than the cable, satellite, Wi-Fi and wireless cellular communication including 5G cellular and cloud computing technologies and derivatives. Herein “*” connotes “diverse and different” and “_n” connotes “large or infinitely large numbers”.

### SUMMARY OF THE INVENTION

CIPS is the innovative computer programs and software for rendering edge-computing and edge-exchanging mobile*_n-to-mobile*_n communication devices by rendering same, similar and/or equivalent digital footprint*_n in, between and among said networked mobile*_n-to-mobile*_n communication devices to rendering individual adaptable edge-computing and edge-exchanging mobile*_n-to-mobile*_n cell*_n and individual adaptably mobile group*_n of adaptable edge-computing and edge-exchanging mobile*_n-to-mobile*_n cell*_n (known as individual*_n, individual group*_n, respectively) and individual*_n in, between and among individual group*_n possessing inherent shifting, expanding, retracting and adaptable networking mobile domains between, among and over place*_n on earth, other planet*_n and outer space (henceforth collectively known as individual*_n and individual group*_n). CIPS for rendering ir3.dividual and individual group*_n-rendered Infinite edge-computing and edge-exchanging Oracle (ECO*_n™) for rendering expanding and adaptable abiotic thinking and reasoning for supplanting present and future random and fragmented thought processes and decisions. Said abiotic thinking and reasoning for preventing, avoiding, tracking, trending and treating definitive and specific continuum*_n of diagnoses of acute, subacute and chronic diseases and disorders and sequelae and related or relevant vital goods, products, services and resources in diverse industries in diverse phases and circumstances of care-giving and long-term care in storage means (ECODI*_n) in lattices of multi-dimensional relational big databases associating with targeted, requesting and rendering networked repositories in, between and among individual networked mobile devices, individual group*_n of networked mobile devices and related networked mobile devices in, between and among said individual group*_n of networked mobile devices (EMC*_n) wherein individual*_n and individual group*_n for instantly and rapidly edge-computing and edge-exchanging relevant continuum*_n and footprint*_n, between and among individual*_n and individual group*_n thence improving, editing, adapting and expanding EMC*_n thence improving, editing, adapting and expanding ECODI*_n thence improving and empowering ECO*_n.

In summary, CIPS rendering ECO*_n-rendered and successively improved and expanded footprint*_n for constantly, instantly and rapidly edge-computing and edge-exchanging integral snippets*_n and relations of integral snippets*_n in said lattices of multi-dimensional relational databases in, between and among individual*_n and individual group*_n.

### DESCRIPTION OF THE PREFERRED EMBODIMENTS

The detailed description of one of the preferred embodiments of the present invention is well described in the Claims of the Invention infra,

The present commercial computer programs and software and Wi-Fi and hot spots for instant and rapid edge-computing and edge-exchanging changing exchange*_n of continuum*_n and footprint*_n, wireless, cellular, near-field and speech recognition technologies including 3G, 4G and 5G cellular, near-field and speech recognition technologies and derivatives suporting the innovative computer programs and software in said storage means attached to said processor units in, between and among said billions of networked mobile*_n-to-mobile*_n repositories in said networked smartphones and laptops and the likes and individual group*_n of servers and stationary communication desktop devices (henceforth collectively known as CIPS). The commercial computer programs and software and technologies supporting said devices include SMS (short message service), Enterprise SMS messaging, also known as application-to-peer messaging (A2P Messaging) or 2-way SMS, radio telegraphy, ANSI, CDMA networks and Digital AMP, social media and derivatives currently installed and applied in billions of smartphones and commercial devices for accepting, processing, sending, receiving and sharing data on in diverse industries as stated in the Claims of the Invention Herein “*” connotes “diverse and different” and “_n” connotes “large or infinitely large numbers”.

Although various preferred embodiments of the present invention have been described for people, it will be appreciated by and obvious to those skilled in the art and science that adaptations, variations and derivatives of said embodiments are and will be made and achieved animals and machines and objects without departing from the spirit and scope of the present invention.

In the present invention, it is understood and acknowledged that sciences, medicine and healthcare applied to various preferred embodiments herein and their adaptations, variations and derivatives without departing from the spirit and scope of the present invention.

1. A system of edge-computing and edge-exchanging mobile*_n-to-mobile*_n communication devices for rendering and successively improving and expanding individual adaptable edge-computing and edge-exchanging mobile*_n-to-mobile*_n cell*_n and individual adaptably mobile group*_n of adaptable edge-computing and edge-exchanging mobile*_n-to-mobile*_n cell*_n (known as individual*_n, individual group*_n, respectively) and individual*_n in, between and among individual group*_n possessing inherent shifting, expanding, retracting and adaptable networking mobile domains between, among and over place*_n on earth, other planet*_n and outer space (henceforth collectively known as adaptable individual*_n and adaptable individual group*_n aka individual*_n and individual group*_n) wherein individual*_n and individual group*_n are networked albeit may not connect to desktop communications devices and servers for cloud computing (henceforth collectively known as EMC*_n) wherein networked repositories for rendering and successively adapting and expanding adaptable Infinite edge-computing and edge-exchanging Oracle for rendering, improving and expanding adaptable abiotic thinking and reasoning for supplanting present and future random and fragmented thought processes and decisions for rendering consequent decisions and related outcomes, occurences and sequalae, and, consequently, Infinite edge-computing and edge-exchanging Oracle for rendering and successively expanding and enhancing said network repositories-rendered abiotic thinking and reasoning of Infinite edge-computing and edge-exchanging Oracle for managing, preventing, avoiding, tracking, trending and treating compositions and compilations of relational databases in storage means of, between and among said networked repositories for rendering and successively and constantly updating, improving and expanding said continuum*_n including digital footprint*_n continuum*_n comprising diagnoses of acute, subacute and chronic diseases and disorders and sequelae and related or relevant vital goods, products, services and resources in diverse industries in diverse phases and circumstances of care-giving, long-term care and living associated with integral snippets*_n and relations of integral snippets*_n comprising relational data on demographics, medical, surgical and related relational data and social histories and data of, between and among users of said devices and identifications and IP addresses of said networked devices in, between and among said continuum*_n including adaptable device*_n digital footprint*_n aka footprint*_n comprising same, similar and/or equivalent integral snippets*_n and relations of same, similar and/or equivalent relational data in, between and among said digital footprint*_n continuum*_n associating with integral snippets*_n and relations of integral snippets*_n associating with demographics, medical and surgical data and social histories of users and individual group*_n of users and identifications and IP addresses and adaptable instantly rendered new real-time and unique or flash or real-time inherent security digital token*_n for protecting against identities thefts, frauds, cyberattacks, hackings and corruptions and rendering fidelity of relational databases in, between and among individual*_n and individual group*_n in EMC*_n (henceforth collectively known as ECO*_n) wherein individual*_n and individual group*_n for using footprint*_n for getting and streaming, pulling, pushing, sharing and disseminating same, similar and/or equivalent footprint*_n in, between and among relevant targeted individual*_n and individual group*_n, requesting individual*_n and individual group*_n and rendering individual*_n and individual group*_n w-herein said same, similar anchor equivalent footprint*_n for rendering and successively updating, improving and expanding individual*_n and individual group*_n based on said same, similar and/or equivalent footprint*_n in said compositions and compilations footprint*_n for rendering and successively editing, updating, adapting and expanding individual*_n and individual group*_n in EMC*_n wherein footprint*_n for rendering and successively editing, updating, adapting and expanding ECODI*_n in EMC*_n thence expanding, retracting and adapting virtual global and space networking domains of individual*_n and individual group*_n in EMC*_n thence rendering and successively editing, updating, adapting and expanding ECO*_n in, between and among storage means (henceforth collectively known as adaptable and expanding ECODI*_n aka ECODI*_n) wherein ECO*_n for rendering and successively updating, improving and expanding digital footprint*_n for rendering and constantly updating, improving and expanding indivdual*_n and individual group*_n ECO*_n in, between and among individual*_n and individual group*_ n for using random snippet*_n of relational data in integral snippets*_n and relations of integral snippets*_n including relational demographics, medical, surgical and vital goods, services, resources data and social histories of users, individual group*_n of users associating with individual group*_n and identifications and IP addresses in said compositions and compilations in. between and among relevant targeted individual*_n and individual group*_n, requesting individual*_n and individual group*_ n and rendering individual*_n and individual group*_n wherein said random snippet*_n of relational data for rendering instant-rendered inherent, new, adaptable and unique or instantly flash or real-time inherent security digital token*_n handshaking between and among networked repositories in, between and among individual*_n and individual group*_n wherein said security digital token*_n for managing, tracking, trending, protecting and fostering said continuum*_n including adaptable device*_n digital footprint*_n in individual*_n and individual group*_n in EMC*_n wherein ECO*_n for rendering and successively improving and expanding said compositions and compilations of adaptable and expanding multi-dimensional electronic and digital lattices of multi-dimensional relational databases comprising integral snippets*_n and relations of integral snippets*_n. in storage means, for rendering and successively editing and updating said compositions and compilations wherein said compositions and compilations comprising continuum*_n include digital footprint continuum*_n comprising same, similar and/or equivalent integral snippets*_n and relations of same, similar and/or equivalent relational data in, between and among said digital footprint continuum*_n associating with integral snippets*_n and relations of integral snippets*_n comprising demographics, medical and surgical data and social histories of users and individual group*_n of users and identifications and IP addresses and adaptable instantly rendered new real-time and unique or flash or real-time inherent security digital token*_n of said networked devices (henceforth collectively known as adaptable device*_n digital footprint*_n aka footprint*_n) thus footprint*_n for rendering and successively editing, updating, adapting and expanding individual*_n and individual group*_n in EMC*_n wherein said networked devices comprising interactive touchscreens including viewing screens (henceforth collectively known as touchscreens), said interactive touchscreens for rendering, updating and expanding interactive said adaptable and expanding compositions and compilations comprising interactive relational data in said integral snippets*_n and relations of integral snippets*_n in ECODI*_n as dynamic lists of interactive adaptable and expanding relational data in said integral snippets*_n and relations of relational data in, between and among integral snippets*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n in EMC*_n wherein said networked repositories-rendered adaptable, changeable, expandable and retractable dynamic interactive multi-dimensional universal network data-driven visualizations comprising interactive universal network data-driven flowingframeworks of interactive visual-hierarchied flowingdata of connected visual-hierarchied data points aka interactive visual-hierachied nodes for rendering, updating and expanding interactive said adaptable and expanding compositions and compilations comprising interactive relational data in said integral snippets*_n, relations of integral snippets*_n and footprint*_n in ECODI*_n as dynamic lists of interactive adaptable and expanding relational data in said integral snippets*_n, relations of relational data and footprint*_n, between and among integral snippets*_n and relations of integral snippets*_n and footprint*_n for rendering, updating and expanding individual*_n and individual group*_n thence rendering, updating and expanding EMC*_n and ECODI*_n thence rendering, updating and expanding ECO*_n in, between and among individual*_n and individual group*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n (henceforth collectively known as interactives) in EMC*_n wherein ECO*_n for rendering and successively editing, updating, adapting, improving and expanding said compositions and compilations in said storage means and as interactive adaptable, selectable and clickable relational data in integral snippets*_n and relations of integral snippets*_n in said continuum*_n including said digital footprint*_n continuum*_n and associated interactive, adaptable, selectable and clickable multi-dimensional universal network data-driven visualizations of said relational data integral snippets*_n and relations of integral snippets*_n in said continuum*_n including said digital footprint*_n continuum*_n for rendering and successively updating, improving and expanding said continuum*_n including adaptable device*_n digital footprint*_n in said networked repositories in individual*_n and individual group*_n and in interactive touchscreens in, between among said networked devices wherein interactives and said speech recognition and near-field technologies for rendering and successively updating, improving and expanding said continuum*_n and said footprint*_n in said compositions and compilations in said storage means, said interactives and footprint*_n individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n. wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n wherein ECO*_n in, between and among individual*_n and individual group*_n-rendered relevant targeted, requesting and rendering networked repositories for rendering integral snippets*_n and relations of integral snippets*_n comprising demographics, medical and surgical goods, products, services and resources data and social histories of users and individual group*_n of users and identifications and IP addresses for rendering adaptable new real-time and unique or flash or real-time inherent security digital token*_n of said networked devices (henceforth collectively known as adaptable device*_n digital footprint*_n aka footprint*_n) thus footprint*_n for rendering and successively editing, updating, adapting and expanding EMC*_n for harnessing and using immense processing powers of billions of said processor units in. between and among individual group*_n of relevant processor units and attached storage means in, between and among individual adaptable group*_n of mobile adaptable edge-computing and edge-exchanging Cell*_n in EMC*_n for rendering and successively adapting, improving and expanding ECODI*_n and ECO*_n in EMC*_n thence rendering and successively adapting, improving and expanding ECO*_n for rendering, improving and expanding adaptable abiotic thinking and reasoning to supplanting present and future random and fragmented thought processes and decisions, ECO*_n thinking and reasoning to managing, tracking and trending said continuum*_n including digital footprint*_n continuum*_n on diagnoses of acute, subacute and chronic diseases and disorders and sequelae and related or relevant vital goods, products, services and resources in diverse industries in diverse phases and circumstances of healthcare, care-giving and long-term care in said compositions and compilations and footprint*_n in, between and among individual*_n and individual group*_n for rendering and successively editing, updating, adapting, improving and expanding ECODI*_n in EMC*_n thence expanding, retracting and adapting virtual global and space networking domains of individual*_n and individual group*_n in EMC*_n, said system comprises:
said mobile*_n-to-mobile*_n communication devices are billions of networked smartphones and laptops comprising said networked repositories comprising said processor units attached to said storage means in individual*_n and individual group*_n based on relational data in said continuum*_n including said digital footprint*_n continuum*_n in said compositions and compilations and footprint*_n, said networked repositories in individual*_n and individual group*_n networked albeit may not connect to desktop communications devices and servers comprising commercial computer programs and software and supported by Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n CIPS for instructing said networked repositories to rendering and successively editing, updating, adapting, improving and expanding said compositions and compilations in said storage means and in, between and among interactives in, between and among individual*_n and individual group*_n and stationary communication desktops and servers wherein interactives and said speech recognition and near-field technologies for rendering and successively updating, improving and expanding said continuum*_n and said footprint*_n in said compositions and compilations in said storage means, said interactives and footprint*_n individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n wherein CIPS for instructing said networked repositories for rendering and successively adapting and expanding adaptable infinite edge-computing and edge-exchanging Oracle for rendering, improving and expanding adaptable abiotic thinking and reasoning for supplanting present and future random and fragmented thought processes and decisions for rendering decisions and related outcomes, occurences and sequalae, and said Infinite edge-computing and edge-exchanging Oracle for rendering and successively expanding and enhancing said network repositories-rendered abiotic, thinking and reasoning for managing, tracking and trending said continuum*_n of diagnoses of acute, subacute and chronic diseases and disorders and sequelae and related or relevant vital goods, products, services and resources in diverse industries in diverse phases and circumstances of healthcare, care-giving and long-term care associating with integral snippets* _n and relations of integral snippets*_n comprising relational data on demographics, medical, surgical and social histories and data of users of EMC*_n and identifications and IP addresses of said networked devices in, between and among said continuum*_n and digital footprint*_n in, between and among said networked repositories wherein CIPS for instructing said networked repositories for rendering said compositions and compilations as interactive selectable and clickable relational data in integral snippets*_n and relations of integral snippets*_n in interactive touchscreens and interactive, selectable and clickable multi-dimensional universal network data-driven visualizations integral snippets*_n and relations of integral snippets*_n in EMC*_n (henceforth collectively known as adaptable and expanding ECODI*_n aka ECODI*_n);
ECO*_n in, between and among individual*_n and individual group*_n for using CIPS for instructing said networked repositories in individual*_n and individual group*_n for rendering instant-rendered inherent, new, adapable and unique or instantly flash or real-time inherent security digital token*_n handshaking between and among individual*_n and individual group*_n wherein said security digital token*_n for managing, tracking, trending, protecting and fostering said continuum*_n including adaptable device*_n digital footprint*_n in individual*_n and individual group*_n in EMC*_n; and
herein “*” connotes “diverse and different” and “_n” connotes “large or infinitely large numbers”.

2. The system of edge-computing and edge-exchanging mobile*_n-to-mobile*_n communication devices according to claim 1 wherein said integral snippets*_n and relations of integral snippets*_n comprising relational data comprise said ECO*_n in, between and among individual*_n and individual group*_n for rendering and successively editing, updating. adapting, improving and expanding industry*_n-specific chronologically sequenced and sequentially time-stamped relevant specialties-, categories- and headings of diagnosis*_n-specific medical history*_n- and surgical*_n-specific differential diagnosis*_n, cause*_n, chief complaints, signs and symptoms, review of systems, physical examination, laboratory tests and results, related, asociated and relevant relational data on individual diagnoses and relational diagnoses and associated differential diagnos*_n, continuum*_n diagnos*_n of acute, subacute and chronic diseases and disorders and sequelae and related or relevant vital goods, products, services and resources in diverse industries in diverse phases and circumstances of healthcare, care-giving and long-term care.

3. The system of edge-computing and edge-exchanging mobile*_n-to-mobile*_n communication devices according to claim 1 wherein adaptable and expanding compositions and compilations of said integral snippets*_n and relations in, between and among said integral snippets*_n in said relational databases comprising continuum*_n including digital footprint*_n continuum*_n of in individual*_n and individual group*_n in EMC*_n, said relational databases comprise relational data of medical, surgical and social specialty-specific, diagnosis-specific, syndrome-specific and medical history specialties-, categories- and headings-specific snippets of relational data of networked repositories-digital id*_n, -timestamped, -temporally and -chronologically ordered relational sequences of present illneses, past medical history, past surgical history, vaccinations, general health, infectious diseases, sources of infections, accidents, injuries, repetitive strain injuries, hospitalizations, social history, substance abuse, drug use, family history, travel history, occupations, avocations, asesments and plans, pharmaceuticals and medications, supplements, allergies, sideeffects and complications, procedures, treatments, therapies, outcomes, comments and feedbacks, associating with relational data in pharmaceuticals, intelligence, pharmaceuticals, financials, insurance, investments, estate plannings, actuarial services, community and social services, acute and chronic long-term, care-giving and end-of-life care, legal services, communication, real estate, travel and transportation, hotel and hospitality industries; in said continuum*_n including digital footprint*_n continuum*_n in said relational databases in, between and among individual*_n and individual group*_n in EMC*_n;

4. The system of edge-computing and edge-exchanging mobile*_n-to-mobile*_n communication devices according to claim 1 wherein said adaptable and expanding integral snippets*_n and relations in, between and among said integral snippets*_n in said relational databases comprise relational words, terms, phrases, graphics, pictures, videos, relational digital contents, phrases, sentences and any combinations thereof on said industries-, specialties-, categories- and headings-specific goods, products, services and resources and related financials and insurance products, services and resources in laypeople's languages and professionals' languages,

5. The system of edge-computing and edge-exchanging mobile*_n-to-mobile*_n communication devices according to claim 1 wherein said interactives comprise continuum*_n including digital footprint*_n continuum*_n on diagnosis*_n of acute, subacute and chronic diseases and disorders and sequelae including metabolic syndrome, metabolism of fats, lipids, lipoproteins, proteins and carbohydrates, coronary atherosclerosis risks (CAR), cardiovascular diseases (CVD) and cerebrovascular diseases and related and associated diagnoses of diseases, disorders and sequelae and related or relevant vital goods, products, services and resources in diverse industries in diverse phases and circumstances of care-giving, long-term care and living.

6. In a system of edge-computing and edge-exchanging mobile*_n-to-mobile*_n communication devices for rendering and successively improving and expanding individual adaptable edge-computing and edge-exchanging mobile*_n-to-mobile*_n cell*_n and individual adaptably mobile group*_n of adaptable edge-computing and edge-exchanging mobile*_n-to-mobile*_n cell*_n (known as individual*_n, individual group*_n, respectively) and individual*_n in, between and among individual group*_n possessing inherent shifting, expanding, retracting and adaptable networking mobile domains between, among and over place*_n on earth, other planet*_n and outer space (henceforth collectively known as adaptable individual*_n and adaptable individual group*_n aka individual*_n and individual group*_n) wherein individual*_n and individual group*_n are networked albeit may not connect to desktop communications devices and servers for cloud computing (henceforth collectively known as EMS*_c) wherein networked repositories for rendering and successively adapting and expanding adaptable Infinite edge-computing and edge-exchanging Oracle for rendering, improving and expanding adaptable abiotic thinking and reasoning for supplanting present and future random and fragmented thought processes and decisions for rendering consequent decisions and related outcomes, occurences and sequalae, and, consequently, Infinite edge-computing and edge-exchanging Oracle for rendering and successively expanding and enhancing said network repositories-rendered abiotic thinking and reasoning of Infinite edge-computing and edge-exchanging Oracle for managing, preventing, avoiding, tracking, trending and treating, compositions and compilations of relational databases in storage means of, between and among said networked repositories for rendering and successively and constantly updating, improving and expanding said continuum*_n including digital footprint*_n continuum*_n comprising diagnoses of acute, subacute and chronic diseases and disorders and sequelae and related or relevant vital goods, products, services and resources in diverse industries in diverse phases and circumstances of care-giving, long-term care and life associated with integral snippets*_n and relations of integral snippets*_n comprising relational data on demographics, medical, surgical and related relational data and social histories and data of, between and among users of said devices and identifications and IP addresses of said networked devices in, between and among said continuum*_n including adaptable device*_n digital footprint*_n aka footprint*_n comprising same, similar and/or equivalent integral snippets*_n and relations of same, similar and/or equivalent relational data in, between and among said digital footprint*_n continuum*_n for rendering and successively improving and expanding individual*_n and individual group*_n (collectively known as adaptable and expanding ECODI*_n aka ECODI*_n) wherein said integral snippets*_n and relations of integral snippets*_n associating with integral snippets*_n and relations of integral snippets*_n associating with demographics, medical and surgical data and social histories of users and individual group*_n of users and identifications and IP addresses and adaptable instantly rendered new real-time and unique or flash or real-tine inherent security digital token*_n for protecting against identities thefts, frauds, cyberattacks, hackings and corruptions and rendering fidelity of relational databases in, between and among individual*_n and individual group*_n in EMC*_n (henceforth collectively known as ECO*_n) wherein ECO*_n for rendering and successively improving and expanding said compositions and compilations of adaptable and expanding multi-dimensional electronic and digital lattices of multi-dimensional relational databases comprising integral snippets and relations of integral snippets*_n, in storage means, for rendering and successively editing and updating said compositions and compilations wherein said compositions and compilations comprising continuum*_n include digital footprint continuum*_n comprising same, similar and/or equivalent integral snippets*_n and relations of same, similar and/or equivalent relational data in, between and among said digital footprint continuum*_n associating with integral snippets*_n and relations of integral snippets*_n comprising demographics, medical and surgical data and social histories of users and individual group*_n of users and identifications and IP addresses and adaptable instantly rendered new real-time and unique or flash or real-time inherent security digital token*_n of said networked devices (henceforth collectively known as adaptable device*_n digital footprint*_n aka footprint*_n) thus footprint*_n for rendering and successively editing, updating, adapting and expanding individual*_n and individual group*_n in EMC*_n wherein said networked devices comprising interactive touchscreens including viewing screens (henceforth collectively known as touchscreens), said interactive touchscreens for rendering, updating and expanding interactive said adaptable and expanding compositions and compilations comprising interactive relational data in said integral snippets*_n and relations of integral snippets*_n in ECODI*_n as dynamic lists of interactive adaptable and expanding relational data in said integral snippets*_n and relations of relational data in, between and among integral snippets*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n in EMC*_n wherein said networked repositories-rendered adaptable, changeable, expandable and retractable dynamic interactive multi-dimensional universal network data-driven visualizations comprising interactive universal network data-driven flowingframeworks of interactive visual-hierarchied flowingdata of connected visual-hierarchied data points aka interactive visual-hierachied nodes for rendering, updating and expanding interactive said adaptable and expanding compositions and compilations comprising interactive relational data in said integral snippets*_n, relations of integral snippets*_n and footprint*_n in ECODI*_n as dynamic lists of interactive adaptable and expanding relational data in said integral snippets*_n, relations of relational data and footprint*_n in, between and among integral snippets*_n and relations of integral snippets*_n and footprint*_n for rendering, updating and expanding individual*_n and individual group*_n thence rendering, updating and expanding EMC*_n and ECODI*_n thence rendering, updating and expanding ECO*_n in, between and among individual*_n and individual group*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n (henceforth collectively known as interactives) in EMC*_n wherein ECO*_n for rendering and successively editing, updating, adapting, improving and expanding said compositions and compilations in said storage means and as interactive adaptable, selectable and clickable relational data in integral snippets*_n relations of integral snippets*_n in said continuum*_n including said digital footprint*_n continuum*_n and associated interactive, adaptable, selectable and clickable multi-dimensional universal network data-driven visualizations of said relational data integral snippets*_n and relations of integral snippets*_n in said continuum*_n including said digital footprint*_n continuum*_n for rendering and successively updating, improving and expanding said continuum*_n including adaptable device*_n digital footprint*_n in said networked repositories in individual*_n and individual group*_n and in interactive touchscreens in, between among said networked devices wherein interactives and said speech recognition and near-field technologies for rendering and successively updating, improving and expanding said continuum*_n and said footprint*_n in said compositions and compilations in said storage means, said interactives and footprint*_n individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n wherein wherein individual*_n and individual group*_n for using footprint*_n for getting and streaming, pulling, pushing, sharing and disseminating same, similar and/or equivalent footprint*_n between and among relevant targeted individual*_n and individual group*_n, requesting individual*_n and individual group*_n and rendering individual*_n and individual group*_n wherein said same, similar and/or equivalent footprint*_n for rendering and successively updating, improving and expanding individual*_n and individual group*_n based on said same, similar and/or equivalent footprint*_n in said compositions and compilations footprint*_n for rendering and successively editing, updating, adapting and expanding individual*_n and individual group*_n EMC*_n wherein footprint*_n for rendering and successively editing, updating, adapting and expanding ECODI*_n in EMC*_n thence expanding, retracting and adapting virtual global and space networking domains of individual*_n and individual group*_n in EMC*_n thence rendering and successively editing, updating, adapting and expanding ECO*_n in, between and among individual*_n and individual group*_n for using CIPS for instructing said networked repositories in, between and among individual*_n and individual group*_n for rendering and successively improving and expanding ECODI*_n and for using random snippet*_n relational data in ECODI*_n in, between and among individual*_n and individual group*_n-rendered relevant targeted, requesting and rendering networked repositories for rendering instant-rendered inherent, new, adapable and unique or instantly flash or real-time inherent security digital token*_n handshaking between and among relevant individual*_n and individual group*_n wherein said security digital token*_n for managing, tracking, trending and protecting ECO*_n and ECODI*_n in said individual*_n and individual group*_n in EMC*_n for harnessing and using immense processing powers of billions of said processor units in, between and among individual group*_n of relevant processor units and attached storage means in, between and among individual adaptable group*_n of mobile adaptable edge-computing and edge-exchanging Cell*_n in EMC*_n for rendering and successively adapting, improving and expanding ECODI*_n and ECO*_n in EMC*_n thence rendering and successively adapting, improving and expanding ECO*_n for rendering, improving and expanding adaptable abiotic thinking and reasoning to supplanting present and future random and fragmented thought processes and decisions, ECO*_n thinking and reasoning to managing, tracking and trending said continuum*_n including digital footprint*_n continuum*_n on diagnoses of acute, subacute and chronic diseases and disorders and sequelae and related or relevant vital goods, products, services and resources in diverse industries in diverse phases and circumstances of healthcare, care-giving and long-term care in said compositions and compilations and footprint*_n in, between and among individual*_n and individual group*_n for rendering and successively editing, updating, apting, improving and expanding ECODI*_n in EMC*_n thence expanding, retracting and adapting virtual global and space networking domains of individual*_n and individual group*_n in EMC*_n said mobile*_n-to-mobile*_n communication devices are billions of networked smartphones and laptops comprising said networked repositories comprising said processor units attached to said storage means in individual*_n and individual group*_n based on relational data in said continuum*_n including said digital footprint*_n continuum*_n in said compositions and compilations and footprint*_n, said networked repositories in individual*_n and individual group*_n networked albeit may not connect to desktop communications devices and servers comprising commercial computer programs and software and supported by Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n, ECO*_n rendering and successively editing, updating, adapting, improving and expanding said compositions and compilations in said storage means and as interactive adaptable, selectable and clickable relational data in integral snippets*_n and relations of integral snippets*_n in said continuum*_n including said digital footprint*_n continuum*_n and associated interactive, adaptable, selectable and clickable multi-dimensional universal network data-driven visualizations of said relational data integral snippets*_n and relations of integral snippets*_n in said continuum*_n including said digital footprint*_n continuum*_n to rendering and successively updating, improving and expanding said continuum*_n including adaptable device*_n digital footprint*_n in said networked repositories in individual*_n and individual group*_n and in interactive touchscreens including viewing screens (henceforth collectively known as touchscreens) in, between among said networked devices wherein interactives and said speech recognition and near-field technologies rendering and successively updating, improving and expanding said continuum*_n and said footprint*_n in said compositions and compilations in said storage means, said interactives and footprint*_n individual*_n and individual group*_n wherein said interactives and said speech recognition and near-field technologies rendering and successively updating, improving and expanding individual*_n and individual group*_n in EMC*_n rendering and successively editing, updating, adapting, improving and expanding ECODI*_n thence rendering and successively editing, updating, adapting, improving and expanding abiotic thinking and reasoning of ECO*_n Thence rendering and successively editing, updating, adapting, improving and expanding individual*_n in and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n rendering and successively editing, updating, adapting and expanding individual*_n and individual group*_n in EMC*_n wherein said networked devices comprising interactive touchscreens including viewing screens (henceforth collectively known as touchscreens), said interactive touchscreens rendering, updating and expanding interactive said adaptable and expanding compositions and compilations comprising interactive relational data in said integral snippets*_n and relations of integral snippets*_n in ECODI*_n as dynamic lists of interactive adaptable and expanding relational data in said integral snippets*_n and relations of relational data in, between and among integral nippets*_n, said touchscreens rendering, adapting and expanding ECODI*_n rendering, updating and expanding EMC*_n and rendering, updating and expanding individual*_n and individual group*_n thence rendering, updating and expanding ECO*_n™ and EMC*_n thence rendering, updating and expanding ECODI*_n rendering and successively adapting and expanding adaptable ECO*_n and EMC*_n purveying and procuring and pushing, pulling and streaming continuum*_n to, between and among relevant targeted and requesting networked repositories and associated touchscreens in, between and among individual*_n and individual group*_n in EMC*_n wherein said networked repositories-rendered adaptable, changeable, expandable and retractable dynamic interactive multi-dimensional universal network data-driven visualizations comprising interactive universal network data-driven flowingframeworks of interactive visual-hierarchied flowingdata of connected visual -hierarchied data points aka interactive visual-hierachied nodes rendering, updating and expanding interactive said adaptable and expanding compositions and compilations comprising interactive relational data in said integral snippets*_n, relations of integral snippets*_n and footprint*_n in ECODI*_n as dynamic lists of interactive adaptable and expanding relational data in said integral snippets*_n, relations of relational data and footprint*_n in, between and among integral snippets*_n and relations of integral snippets*_n and footprint*_n rendering, updating and expanding individual*_n and individual group*_n thence rendering, updating and expanding EMC*_n and ECODI*_n thence rendering, updating and expanding ECO*_n in, between and among individual*_n and individual group*_n, said touchscreens rendering, adapting and expanding ECODI*_n rendering, updating and expanding EMC*_n and rendering, updating and expanding individual*_n and individual group*_n thence rendering, updating and expanding ECO*_n™ and EMC*_n thence rendering, updating and expanding ECODI*_n rendering and successively adapting and expanding adaptable ECO*_n and EMC*_n purveying and procuring and pushing, pulling and streaming continuum*_n to, between and among relevant targeted and requesting networked repositories and associated touchscreens in, between and among individual*_n and individual group*_n (henceforth collectively known as interactives) in EMC*_n wherein CIPS for instructing said networked repositories to rendering and successively editing, updating, adapting, improving and expanding said compositions and compilations in said storage means and in, between and among interactives in, between and among individual*_n and individual group*_n and stationary communication desktops and servers wherein interactives and said speech recognition and near-field technologies for rendering and successively updating, improving and expanding said continuum*_n and said footprint*_n in said compositions and compilations in said storage means, said interactives and footprint*_n individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n wherein CIPS for instructing said networked repositories for rendering and successively adapting and expanding adaptable Infinite edge-computing and edge-exchanging Oracle for rendering, improving and expanding adaptable abiotic thinking and reasoning for supplanting present and future random and fragmented thought processes and decisions for rendering decisions and related outcomes, occurences and sequalae, and said Infinite edge-computing and edge-exchanging Oracle for rendering and successively expanding and enhancing said network repositories-rendered abiotic thinking and reasoning for managing, tracking and trending said continuum*_n of diagnoses of acute, subacute and chronic diseases and disorders and sequelae and related or relevant vital goods, products, services and resources in diverse industries in diverse phases and circumstances of healthcare, care-giving and long-term care associating with integral snippets*_n and relations of integral snippets*_n comprising relational data on demographics, medical, surgical and social histories and data of users of EMC*_n and identifications and IF addresses of said networked devices in, between and among said continuum*_n and digital footprint*_n in, between and among said networked repositories wherein CIPS for instructing said networked repositories for rendering said compositions and compilations as interactive selectable and clickable relational data in integral snippets*_n and relations of integral snippets*_n in interactive touchscreens and interactive, selectable and clickable multi-dimensional universal network data-driven visualizations integral snippets*_n and relations of integral snippets*_n in EMC*_n (henceforth collectively known as adaptable and expanding ECODI*_n aka ECODI*_n);
ECO*_n in, between and among individual*_n and individual group*_n for using CIPS for instructing said networked repositories in individual*_n and individual group*_n for rendering instant-rendered inherent, new, adapable and unique or instantly flash or real-time inherent security digital token*_n handshaking between and among individual*_n and individual group*_n wherein said security digital token*_n for managing, tracking, trending and protecting said individual*_n and individual group*_n in EMC*_n, a method of edge-computing and edge-exchanging mobile*_n-to-mobile*_n communication devices rendering and successively improving and expanding individual adaptable edge-computing and edge-exchanging individual*_n and individual group*_n in EMC*_n wherein networked repositories rendering and successively adapting and expanding ECO*_n to rendering, improving and expanding said adaptable abiotic thinking and reasoning to supplanting present and future random and fragmented thought processes and decisions so, consequently, to rendering definitive and specific decisions and related outcomes, occurences and sequalae and ECO*_n rendering and successively expanding and enhancing said network repositories-rendered abiotic thinking and reasoning of ECO*_n to managing, preventing, avoiding, tracking, trending and treating compositions and compilations of relational databases in. storage means of, between and among said networked repositories rendering and successively and constantly updating, improving and expanding said continuum*_n including digital footprint*_n continuum*_n comprising diagnoses of acute, subacute and chronic diseases and disorders and sequelae and related or relevant vital goods, products, services and resources in diverse industries in diverse phases and circumstances of care-giving, long-term care and life associating with integral snippets*_n and relations of integral snippets*_n comprising relational data on demographics, medical, surgical and related relational data and social histories and data of, between and among users of said devices and identifications and IP addresses of said networked devices in, between and among said continuum*_n including footprint*_n comprising same, similar and/or equivalent integral snippets*_n and relations of same, similar and/or equivalent relational data in, between and among digital footprint*_n continuum*_n associating with integral snippets*_n and relations of integral snippets*_n associating with demographics, medical and surgical data and social histories of users and individual group*_n of users and identifications and IP addresses and adaptable instantly rendered new real-time and unique or flash or real-time inherent security digital token*_n to protecting against identities thefts, frauds, cyberattacks, hackings and corruptions of and rendering fidelity of relational databases in, between and among individual*_n and individual group*_n in EMC*_n (collectively known herein as ECO*_n, said “*” connotes “diverse and different” and “_n” connotes “large or infinitely large numbers”, said method comprises processes of:
installing CIPS as app, apps or otherwise in, between and among said networked repositories in mobile*_n-to-mobile*_n communication devices which are billions of networked smartphones and laptops comprising said networked repositories comprising said processor units attached to said storage means in individual*_n and individual group*_n based on relational data in said continuum*_n including said digital footprint*_n continuum*_n in said compositions and compilations and footprint*_n, said networked repositories in individual*_n and individual group*_n networked albeit may connect to desktop communications devices and servers comprising commercial computer programs and software and supported by Wi-Fi and hot spots for instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n to rendering and successively editing, updating, adapting and expanding individual*_n and individual group*_n in EMC*_n to individual*_n and individual group*_n using footprint*_n to getting and streaming, pulling, pushing, sharing and disseminating same, similar and/or equivalent footprint*_n in, between and among relevant targeted individual*_n and individual group*_n, requesting individual*_n and individual group*_n and rendering individual*_n and individual group*_n whereby said same, similar and/or equivalent footprint*_n rendering and successively updating, improving and expanding individual*_n and individual group*_n possessing and sharing said same, similar and/or equivalent footprint*_n in said compositions and compilations footprint*_n rendering and successively editing, updating, adapting and expanding individual*_n and individual Group n in EMC*_n wherein footprint*_n to rendering and successively editing, updating, adapting and expanding ECODI*_n in EMC*_n thence expanding, retracting and adapting virtual global and space networking domains of individual*_n and individual group*_n in EMC*_n thence rendering and successively editing, updating, adapting and expanding ECO*_n and ECODI*_n in, between and among storage means whereby ECO*_n to rendering and successively updating, improving and expanding digital footprint*_n to rendering and constantly updating, improving and expanding ECO*_n and ECODI*_n in indivdual*_n and individual group*_n ECO*_n in, between and among individual*_n and individual group*_n using random snippet*_n of relational data in integral snippets*_n and relations of integral snippets*_n including relational demographics, medical surgical and vital goods, services, resources data and social histories of users, individual group*_n of users associating with individual group*_n and identifications and IP addresses in said compositions and compilations in, between and among relevant targeted individual*_n and individual group*_n, requesting individual*_n and individual group*_n and rendering individual*_n and individual group*_n wherein said random snippet*_n of relational data to rendering instant-rendered inherent, new, adapable and unique or instantly flash or real-time inherent security digital token*_n handshaking between and among networked repositories in, between and among individual*_n and individual group*_n wherein said security digital token*_n to managing, tracking, trending, protecting and fostering said continuum*_n including adaptable device*_n digital footprint*_n in, between and among individual*_n and individual group*_n in EMC*_n wherein ECO*_n in, between and among individual*_n and individual group*_n-rendered relevant targeted, requesting and rendering networked repositories rendering integral snippets*_n and relations of integral snippets*_n comprising demographics, medical and surgical goods, products, services and resources data and social histories of users and individual group*_n of users and identifications and IP addresses to rendering adaptable new real-time and unique or flash or real-time inherent security digital token*_n of said networked devices (footprint*_n) thus footprint*_n to rendering and successively editing, updating, adapting and expanding EMC*_n to harnessing and using immense processing powers of billions of said processor units in. between and among individual group*_n of relevant processor units and attached storage means in, between and among individual adaptable group*_n of mobile adaptable edge-computing and edge-exchanging Cell*_n in EMC*_n to rendering and successively adapting, improving and expanding ECODI*_n and ECO*_n in EMC*_n thence rendering and successively adapting, improving and expanding ECO*_n to rendering, improving and expanding adaptable abiotic thinking and reasoning to supplanting present and future random and fragmented thought processes and decisions. ECO*_n thinking and reasoning to managing, tracking and trending said continuum*_n including digital footprint*_n continuum*_n on diagnoses of acute, subacute and chronic diseases and disorders and sequelae and related or relevant vital goods, products, services and resources in diverse industries in diverse phases and circumstances of healthcare, care-giving and long-term care in said compositions and compilations and footprint*_n in, between and among individual*_n and individual group*_n to rendering and successively editing, updating, adapting, improving and expanding ECODI*_n in EMC*_n thence expanding, retracting and adapting virtual global and space networking domains of individual*_ n and individual group*_n in EMC*_n whereby CIPS instructing individual*_n and individual group*_n to rendering and successively editing, updating, adapting, improving and expanding said compositions and compilations in said storage means and in, between and among interactives in. between and among individual*_n and individual group*_n and stationary communication desktops and servers wherein interactives and said speech recognition and near-field technologies rendering and successively updating, improving and expanding said continuum*_n and said footprint*_n in said compositions and compilations in said storage means, said interactives and footprint*_n in, between and among individual*_n and individual group*_n wherein said interactives and said speech recognition and near-field technologies rendering and successively updating, improving and expanding individual*_n and individual group*_n in EMC*_n to rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n to rendering and successively editing, updating, adapting and expanding individual*_n and individual group*_n in EMC*_n -whereby CAPS instructing said networked repositories to rendering and successively adapting and expanding adaptable Infinite edge-computing and edge-exchanging Oracle to rendering, improving and expanding adaptable abiotic thinking and reasoning to supplanting present and future random and fragmented thought processes and decisions to rendering decisions and related outcomes, occurences and sequalae, and said Infinite edge-computing and edge-exchanging Oracle to rendering and successively expanding and enhancing said network repositories-rendered abiotic thinking and reasoning to managing, tracking and trending said continuum*_n of diagnoses of acute, subacute and chronic diseases and disorders and sequelae and related or relevant vital goods, products, services and resources in diverse industries in diverse phases and circumstances of healthcare, care-giving and long-term care associating with integral snippets*_n and relations of integral snippets*_n comprising relational data on demographics, medical, surgical and social histories and data of users of EMC*_n and identifications and IP addresses of said networked devices in, between and among said continuum*_n and digital footprint*_n in, between and among said networked repositories wherein CIPS instructing said networked repositories to rendering said compositions and compilations as interactive selectable and clickable relational data in integral snippets*_n and relations of integral snippets*_n in interactive touchscreens and interactive, selectable and clickable multi-dimensional. universal network data-driven visualizations integral snippets*_n and relations of integral snippets*_n in EMC*_n (henceforth collectively known as adaptable and expanding ECODI*_n aka ECODI*_n);
ECO*_n in, between and among individual*_n and individual group*_n using CIPS to instructing said networked repositories to getting and streaming, pulling, pushing, sharing and disseminating same, similar and/or equivalent relational data in integral snippets*_n and relations of integral snippets*_n in said continuum*_n of digital footprint*_n continuum*_n in said compositions and compilations to rendering and successively updating, improving and expanding said footprint*_n in, between and among relevant targeted, requesting and renderingindividual*_n and individual group*_n to rendering and successively editing, updating, improving and expanding EMC*_n thence expanding, retracting and adapting virtual global and space networking domains of EMC*_n thence rendering and successively editing, updating, improving and expanding ECODI*_n and ECO*_n; and
ECO*_n in, between and among individual*_n and individual group*_n using CIPS to instructing said networked repositories in individual*_n and individual group*_n to rendering instant-rendered inherent, new, adapable and unique or instantly flash or real-time inherent security digital token*_n handshaking between and among individual*_n and individual group*_n wherein said security digital token*_n to managing, tracking, trending, protecting and fostering said continuum*_n including adaptable device*_n digital footprint*_n in individual*_n and individual group*_n in EMC*_n.

7. The process according to claim 6 wherein said integral snippets*_n and relations of integral snippets*_n comprising relational data comprises said ECO*_n in, between and among individual*_n and individual group*_n to rendering and successively editing, updating, adapting, improving and expanding industry*_n-specific chronologically sequenced and sequentially time-stamped relevant specialties-, categories- and headings of diagnosis*_n-specific medical history*_n and surgical*_n-specific differential diagnosis*_n, cause*_n, chief complaints, signs and symptoms, review of systems, physical examination, laboratory tests and results, related, asociated and relevant relational data on individual diagnoses and relational diagnoses and associated differential diagnoses related to continuum*_n on diagnoses of acute, subacute and chronic diseases and disorders and sequelae and related or relevant vital goods, products, services and resources in diverse industries in diverse phases and circumstances of healthcare care-giving and long-term care.

8. The process according to claim 6 wherein edge-computing and edge-exchanging mobile*_n-to-mobile*_n communication devices rendering and successively editing, updating, adapting, improving and expanding compositions and compilations of said integral snippets I and relations in, between and among said integral snippets*_n in said relational databases comprising ECO*_n rendering and successively editing, updating, adapting, improving and expanding continuum*_n including digital footprint*_n continuum*_n in, between and among individual*_n and individual group*_n in EMC*_n, said relational databases comprises relational data of medical, surgical and social specialty-specific, diagnosis-specific, syndrome-specific and medical history specialties-, categories- and headings-specific snippets of relational data of networked repositories-digital id*_n, -timestamped, -temporally and -chronologically ordered relational sequences of present illneses, past medical history, past surgical history, vaccinations, general health, infectious diseases, sources of infections, accidents, injuries, repetitive strain injuries, hospitalizations, social history, substance abuse, recreation drug use, family history, travel history, occupations, avocations, asesments and plans, pharmaceuticals and medications, supplements, allergies, sideeffects and complications, procedures, treatments, therapies, outcomes, comments and feedbacks, associating with relational data in pharmaceuticals, intelligence, pharmaceuticals, financials, insurance, investments, estate plannings, actuarial services, community and social services, acute and chronic long-term, care-giving and end-of-life care, legal services, communication, real estate. travel and transportation, hotel and hospitality industries in said continuum*_n including digital footprint*_n continuum*_n in said relational databases in, between and among individual*_n and individual group*_n in EMC*_n.

9. The process according to claim 6 wherein ECO*_n rendering and successively editing, updating, adapting, improving and expanding said adaptable and expanding integral snippets*_n and relations in, between and among said integral snippets*_n in said relational databases comprises ECO*_n rendering and successively editing, updating, adapting, improving and expandingrelational words, terms, phrases, graphics, pictures, videos, relational digital contents, phrases, sentences and any combinations thereof on said industries-, specialties-, categories- and headings-specific goods, products, services and resources and related financials and insurance products, services and resources in laypeople's languages and professionals' languages.

10. The process according to claim 6 wherein said interactives rendering and successively editing, updating, adapting, improving and expanding said adaptable and expanding continuum*_n including digital footprint*_n continuum*_n on diagnosis*_n of acute, subacute and chronic diseases and disorders and sequelae including metabolic syndrome, metabolism of fats, lipids, lipoproteins, proteins and carbohydrates, coronary atherosclerosis risks (CAR), cardiovascular diseases (CVD) and cerebrovascular diseases and related and associated diagnoses of diseases, disorders and sequelae and related or relevant vital goods, products, services and resources in diverse industries in diverse phases and circumstances of care-giving, long-term care and living.

11. In a system of edge-computing and edge-exchanging mobile*_n-to-mobile*_n communication devices for rendering and successively improving and expanding individual adaptable edge-computing and edge-exchanging mobile*_n-to-mobile*_n cell'*_n and individual adaptably mobile group*_n of adaptable edge-computing and edge-exchanging mobile*_n-to-mobile*_n cell*_n (known as individual*_n, individual group*_n, respectively) and individual*_n in, between and among individual group*_n possessing inherent shifting, expanding, retracting and adaptable networking mobile domains (henceforth known as adaptable individual*_n and adaptable individual group*_n aka individual*_n and individual group*_n) wherein networked repositories in individual*_n and individual group*_n are networked albeit may not connect to desktop communications devices and servers in, between and among place*_n on earth, other planet*_n and outer space (henceforth collectively known as EMC*_n) wherein networked repositories for rendering and successively adapting and expanding adaptable infinite edge-computing and edge-exchanging Oracle for rendering, improving and expanding adaptable abiotic thinking and reasoning for supplanting present and future random and fragmented thought processes and decisions for rendering consequent decisions and related outcomes, occurences and sequalae, and, consequently, Infinite edge-computing and edge-exchanging Oracle for rendering and successively expanding and enhancing said network repositories-rendered abiotic thinking and reasoning of Infinite edge-computing and edge-exchanging Oracle for managing, preventing, avoiding, tracking, trending and treating compositions and compilations of relational databases in storage means of, between and among said networked repositories for rendering and successively and constantly updating, improving and expanding said continuum*_n including digital footprint*_n continuum*_n comprising diagnoses of acute, subacute and chronic diseases and disorders and sequelae and related or relevant vital goods, products, services and resources in diverse industries in diverse phases and circumstances of care-giving, long-term care and life associated with integral snippets*_n and relations of integral snippets*_n comprising relational data on demographics, medical, surgical and related relational data and social histories and data of, between and among users of said devices and identifications and IP addresses of said networked devices in, between and among said continuum*_n including adaptable device*_n digital footprint*_n aka footprint*_n comprising same, similar and/or equivalent integral snippets*_n and relations of same, similar and/or equivalent relational data in, between and among said digital footprint*_n continuum*_n for rendering and successively improving and expanding individual*_n and individual group*_n (collectively known as adaptable and expanding ECODI*_n aka ECODI*_n) wherein said integral snippets*_n and relations of integral snippets*_n associating with integral snippets*_n and relations of integral snippets*_n associating with demographics, medical and surgical data and social histories of users and individual group*_n of users and identifications and IP addresses and adaptable instantly rendered new real-time and unique or flash or real-time inherent security digital token*_n for protecting against identities thefts, frauds, cyberattacks, hackings and corruptions and rendering fidelity of relational databases in, between and among individual*_n and individual group*_n in EMC*_n (henceforth collectively known as ECO*_n) wherein ECO*_n for rendering and successively improving and expanding said compositions and compilations of adaptable and expanding multi-dimensional electronic and digital lattices of multi-dimensional relational databases comprising integral snippets*_n and relations of integral snippets*_n, in storage means, for rendering and successively editing and updating said compositions and compilations wherein said compositions and compilations comprising continuum*_n include digital footprint continuum*_n comprising same, similar and/or equivalent integral snippets*_n and relations of same, similar and/or equivalent relational data in, between and among said digital footprint continuum*_n associating with integral snippets*_n and relations of integral snippets*_n comprising demographics, medical and surgical data and social histories of users and individual group*_n of users and identifications and IP addresses and adaptable instantly rendered new real-time and unique or flash or real-time inherent security digital token*_n of said networked devices (henceforth collectively known as adaptable device*_n digital footprint*_n aka footprint*_n) thus footprint*_n for rendering and successively editing, updating, adapting and expanding individual*_n and individual group*_n in EMC*_n wherein ECO*_n for rendering and successively editing, updating, adapting, improving and expanding said compositions and compilations in said storage means and as interactive adaptable, selectable and clickable relational data in integral snippets*_n and relations of integral snippets*_n in said continuum*_n including said digital footprint*_n continuum*_n and associated interactive, adaptable, selectable and clickable multi-dimensional universal network data-driven visualisations of said relational data integral snippets*_n and relations of integral snippets*_n in said continuum*_n including said digital footprint*_n continuum*_n for rendering and successively updating, improving and expanding said continuum*_n including adaptable device*_n digital footprint*_n in said networked repositories in individual*_n and individual group*_n and in interactive touchscreens including viewing screens (henceforth collectively known as touchscreens) in, between among said networked devices wherein interactives and said speech recognition and near-field technologies for rendering and successively updating, improving and expanding said continuum*_n and said footprint*_n in said compositions find compilations in said storage means, said interactives and footprint*_n individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n wherein said networked devices comprising interactive touchscreens including viewing screens (henceforth collectively known as touchscreens), said interactive touchscreens for rendering, updating and expanding interactive said adaptable and expanding compositions and compilations comprising interactive relational data in said integral snippets*_n and relations of integral snippets*_n in ECODI*_n as dynamic lists of interactive adaptable and expanding relational data in said integral snippets*_n and relations of relational data in, between and among integral snippets*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n in EMC*_n wherein said networked repositories-rendered adaptable, changeable, expandable and retractable dynamic interactive multi-dimensional universal network data-driven visualizations comprising interactive universal network data-driven flowingframeworks of interactive visual-hierarchied flowingdata of connected visual-hierarchied data points aka interactive visual-hierachied nodes for rendering, updating and expanding interactive said adaptable and expanding compositions and compilations comprising interactive relational data in said integral snippets*_n, relations of integral snippets*_n and footprint*_n in ECODI*_n as dynamic lists of interactive adaptable and expanding relational data in said integral snippets*_n, relations of relational data and footprint*_n in, between and among integral snippets*_n and relations of integral snippets*_n and footprint*_n for rendering, updating and expanding individual*_n and individual group*_n thence rendering, updating and expanding EMC*_n and ECODI*_n thence rendering, updating and expanding ECO*_n in, between and among individual*_n and individual group*_n, said touchscreens for rendering, adapting and expanding ECODI*_n for rendering, updating and expanding EMC*_n and for rendering, updating and expanding individual*_n and individual group*_n thence rendering, updating and expanding ECO*_n™ and EMC*_n thence rendering, updating and expanding ECODI*_n for rendering and successively adapting and expanding adaptable ECO*_n and EMC*_n for purveying and procuring and for pushing, pulling and streaming continuum*_n to, between and among relevant targeted and requesting networked repositories and associated touchscreens in, between and among individual*_n and individual group*_n (henceforth collectively known as interactives) in EMC*_n wherein wherein individual*_n and individual group*_n for using footprint*_n for getting and streaming, pulling, pushing, sharing and disseminating same, similar and/or equivalent footprint*_n in, between and among relevant targeted individual*_n and individual group*_n, requesting individual*_n and individual group*_n and rendering individual*_n and individual group*_n wherein said same, similar and/or equivalent footprint*_n for rendering and successively updating, improving and expanding individual*_n and individual group*_n based on said same, similar and/or equivalent footprint*_n in said compositions and compilations footprint*_n for rendering and successively editing, updating, adapting and expanding individual*_n and individual group*_n in EMC*_n wherein footprint*_n for rendering and successively editing, updating, adapting and expanding ECODI*_n in EMC*_n thence expanding, retracting and adapting virtual global and space networking domains of individual*_n and individual group*_n in EMC*_n thence rendering and successively editing, updating, adapting and expanding ECO*_n in, between and among individual*_n and individual group*_n for using CIPS for instructing said networked repositories in, between and among individual*_n and individual group*_n for rendering and successively improving and expanding ECODI*_n and for using random snippet*_n relational data in ECODI*_n in, between and among individual*_n and individual group*_n-rendered relevant targeted, requesting and rendering networked repositories for rendering instant-rendered inherent, new, adapable and unique or instantly flash or real-time inherent security digital token*_n handshaking between and among relevant individual*_n and individual group*_n wherein said security digital token*_n for managing, tracking, trending and protecting ECO*_n and ECODI*_n in said individual*_n and individual group*_n in EMC*_n for harnessing and using immense processing powers of billions of said processor units in, between and among individual group*_n of relevant processor units and attached storage means in, between and among individual adaptable group*_n of mobile adaptable edge-computing and edge-exchanging Cell*_n in EMC*_n for rendering and successively adapting, improving and expanding ECODI*_n and ECO*_n in EMC*_n thence rendering and successively adapting, improving and expanding ECO*_n for rendering, improving and expanding adaptable abiotic thinking and reasoning to supplanting present and future random and fragmented thought processes and decisions, ECO*_n thinking and reasoning to managing, tracking and trending said continuum*_n including digital footprint*_n continuum*_n on diagnoses of acute, subacute and chronic diseases and disorders and sequelae and related or relevant vital goods, products, services and resources in diverse industries in diverse phases and circumstances of healthcare, care-giving and long-term care in said compositions and compilations and footprint*_n in, between and among individual*_n and individual group*_n for rendering and successively editing, updating, adapting, improving and expanding ECODI*_n in EMC*_n thence expanding, retracting and adapting virtual global and space networking domains of individual*_n and individual group*_n in EMC*_n;
said mobile*_n-to-mobile*_n communication devices are billions of networked smartphones and laptops comprising said networked repositories comprising said processor units attached to said storage means in individual*_n and individual group*_n based on relational data in said continuum*_n including said digital footprint*_n continuum*_n in said compositions and compilations and footprint*_n, said networked repositories in individual*_n and individual group*_n networked albeit may not connect to desktop communications devices and servers comprising commercial computer programs and software and supported by Wi-Fi and hot spots for instant and rapid edge-computing and edge-exchanging exchange*_n of continuum*_n and footprint*_n, wireless and cellular technologies and derivatives and speech recognition and near-field technologies for rendering and successively editing, updating, adapting and expanding footprint*_n for rendering and successively editing, updating, adapting and expanding individual*_n and individual group*_n in EMC*_n;
CIPS for instructing said networked repositories to rendering and successively editing, updating, adapting, improving and expanding said compositions and compilations in said storage means and in, between and among interactives in, between and among individual*_n and individual group*_n and stationary communication desktops and servers wherein interactives and said speech recognition and near-field technologies for rendering and successively updating, improving and expanding said continuum*_n and said footprint*_n in said compositions and compilations in said storage means, said interactives and footprint*_n in, between and among individual*_n and individual group*_n wherein said interactives and said speech recognition and near-field technologies for rendering and successively updating, improving and expanding individual*_n and individual group*_n in EMC*_n for rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and
derivatives and speech recognition and near-field technologies for rendering and successively editing, updating, adapting and expanding footprint*_n for rendering and successively editing, updating, adapting and expanding individual*_n and individual group*_n in EMC*_n wherein CIPS for instructing said networked repositories for rendering and successively adapting and expanding adaptable Infinite edge-computing and edge-exchanging Oracle for rendering, improving and expanding adaptable abiotic thinking and reasoning for supplanting present and future random and fragmented thought processes and decisions for rendering decisions and related outcomes, occurences and sequalae, and said Infinite edge-computing and edge-exchanging Oracle for rendering and successively expanding and enhancing said network repositories-rendered abiotic thinking and reasoning for managing, tracking and trending said continuum*_n of diagnoses of acute, subacute and chronic diseases and disorders and sequelae and related or relevant vital goods, products, services and resources in diverse industries in diverse phases and circumstances of healthcare, care-giving and long-term care associating with integral snippets*_n and relations of integral snippets*_n comprising relational data on demographics, medical, surgical and social histories and data of users of EMC*_n and identifications and IP addresses of said networked devices in, between and among said continuum*_n and digital footprint*_n in, between and among said networked repositories wherein. CIPS for instructing said networked repositories for rendering said compositions and compilations as interactive selectable and clickable relational data in integral snippets*_n and relations of integral snippets*_n in interactive touchscreens and interactive, selectable and clickable multi-dimensional universal network data-driven visualizations integral snippets*_n and relations of integral snippets*_n in EMC*_n (henceforth collectively known as adaptable and expanding ECODI*_n aka ECODI*_n); and
ECO*_n in, between and among individual*_n and individual group*_n for using CIPS for instructing said networked repositories in individual*_n and individual group*_n for rendering instant-rendered inherent, new, adaptable and unique or instantly flash or real-time inherent security digital token*_n handshaking between and among individual*_n and individual group*_n wherein said security digital token*_n for managing, tracking, trending and protecting said individual*_n and individual group*_n in EMC*_n, a method of ECO*_n rendering edge-computing and edge-exchanging mobile*_n-to-mobile*_n communication devices comprising individual*_n and individual group*_n to rendering and successively updating, improving and expanding ECODI*_n thence rendering and successively updating, improving and expanding individual*_n and individual group*_n in EMC*_n wherein ECO*_n rendering and successively updating, improving and expanding interactives being assisted and complemented by said speech recognition and near-field technologies to rendering and successively updating, improving and expanding individual*_n and individual group*_n to rendering said interactives to rendering and successively updating, improving and expanding said continuum*_n and footprint*_n in, between and among individual*_n and individual group*_n in said compositions and compilations in said storage means wherein individual*_n and individual group*_n having same, similar and/or equivalent footprint*_n in ECODI*_n which rendering and successively editing, updating, adapting, improving and expanding abiotic thinking and reasoning of ECO*_n in individual*_n and individual group*_n being supported by commercial computer programs and software and Wi-Fi and hot spots for instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n in, between and among individual*_n and individual group*_n rendering and successively editing, updating, adapting and expanding individual*_n and individual group*_n in EMC*_n and ECODI*_n thence rendering and successively updating, improving and expanding individual*_n and individual group*_n in EMC*_n thence rendering and successively updating, improving and expanding said networked repositories-rendered abiotic thinking and reasoning of ECO*_n to supplanting present and future random and fragmented thought processes and decisions to rendering consequent decisions and related outcomes, occurences and sequalae, and, consequently, ECO*_n to rendering and successively expanding and enhancing said network repositories-rendered abiotic thinking and reasoning of ECO*_n to managing, preventing, avoiding, tracking, trending and treating compositions and compilations of relational databases in storage means of, between and among individual*_n and individual group*_n rendering and successively and constantly updating, improving and expanding said continuum*_n including digital footprint*_n continuum*_n associated with integral snippets*_n and relations of integral snippets*_n comprising relational data on demographics, medical, surgical and related relational data and social histories and data of between and among users of said devices and identifications and IP addresses of said networked devices in, between and among said continuum*_n including footprint*_n and individual*_n and individual group*_n-rendered new real-time and unique or flash or real-time inherent security digital token*_n to protecting against identities thefts, frauds, cyberattacks, hackings and corruptions and rendering fidelity of relational data, said method comprises processes of:
downloading C1PS in said networked repositories in said mobile*_n-to-mobile*_n communication devices possessing commercial computer programs and software supported by and hot spots to instant and rapid edge-computing and edge-exchanging exchange*_n of continuum'in. and footprint*_n, wireless and cellular technologies and derivatives and speech recognition and near-field technologies and presently networked to stationary communication devices and servers;
CIPS instructing individual*_n and individual group* to rendering and successively editing, updating, adapting, improving and expanding said compositions and compilations in said storage means and in, between and among interactives in, between and among individual*_n and individual group*_n and stationary communication desktops and servers wherein interactives and said speech recognition and near-field technologies rendering and successively updating, improving and expanding said continuum*_n and said footprint*_n in said compositions and compilations in said storage means, said interactives and footprint*_n individual*_n and individual group*_n wherein said interactives and said speech recognition and near-field technologies rendering and successively updating, improving and expanding individual*_n and individual group*_n in EMC*_n to rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots to instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n to rendering and successively editing, updating, adapting and expanding individual*_n and individual group*_n in EMC*_n whereby ECO*_n rendering and successively editing, updating, adapting, improving and expanding said compositions and compilations in said storage means and as interactive adaptable, selectable and clickable relational data in integral snippets*_n and relations of integral snippets*_n in said continuum*_n including said digital footprint*_n continuum*_n and associated interactive, adaptable, selectable and clickable multi-dimensional universal network data-driven visualizations of said relational data integral snippets*_n and relations of integral snippets*_n in said continuum*_n including said digital footprint*_n continuum*_n to rendering and successively updating, improving and expanding said continuum*_n including adaptable device*_n digital footprint*_n in said networked repositories in individual*_n and individual group*_n and in interactive touchscreens including viewing screens (henceforth collectively known as touchscreens) in, between among said networked devices wherein interactives and said speech recognition and near-field technologies rendering and successively updating, improving and expanding said continuum*_n and said footprint*_n in said compositions and compilations in said storage means, said interactives and footprint*_n individual*_n and individual group*_n wherein said interactives and said speech recognition and near-field technologies rendering and successively updating, improving and expanding individual*_n and individual group*_n in EMC*_n rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-fi and hot spots to instant and rapid edge-computing and edge-exchanging exchange*_n of continuum*_n and footprint*_n, wireless and cellular technologies and derivatives and speech recognition and near-field technologies rendering and successively editing, updating, adapting and expanding footprint*_n rendering and successively editing, updating, adapting and expanding individual*_n and individual group*_n in EMC*_n wherein said networked devices comprising interactive touchscreens including viewing screens (henceforth collectively known as touchscreens), said interactive touchscreens rendering, updating and expanding interactive said adaptable and expanding compositions and compilations comprising interactive relational data in said integral snippets*_n and relations of integral snippets*_n in ECODI*_n as dynamic lists of interactive adaptable and expanding relational data in said integral snippets*_n and relations of relational data in, between and among integral snippets*_n, said touchscreens rendering, adapting and expanding ECODI*_n rendering, updating and expanding EMC*_n and rendering, updating and expanding individual*_n and individual group*_n thence rendering, updating and expanding ECO*_n™ and EMC*_n thence rendering, updating and expanding ECODI*_n rendering and successively adapting and expanding adaptable ECO*_n and EMC*_n purveying and procuring and pushing, pulling and streaming continuum*_n to, between and among relevant targeted and requesting networked repositories and associated touchscreens in, between and among individual*_n and individual group*_n in EMC*_n wherein said networked repositories-rendered adaptable, changeable, expandable and retractable amic interactive multi-dimensional universal network data-driven visualizations comprising interactive universal network data-driven flowingframeworks of interactive visual-hierarchied flowingdata of connected visual-hierarchied data points aka interactive visual-hierachied nodes rendering, updating and expanding interactive said adaptable and expanding compositions and compilations comprising interactive relational data in said integral snippets*_n, relations of integral snippets*_n and footprint*_n in ECODI*_n as dynamic lists of interactive adaptable and expanding relational data in said integral snippets*_n, relations of relational data and footprint*_n in, between and among integral snippets*_n and relations of integral snippets*_n and footprint*_n rendering, updating and expanding individual*_n and individual group*_n thence rendering, updating and expanding EMC*_n and ECODI*_n thence rendering, updating and expanding ECO*_n in, between and among individual*_n and individual group*_n, said touchscreens rendering, adapting and expanding ECODI*_n rendering, updating and expanding EMC*_n and rendering, updating and expanding individual*_n and individual group*_n thence rendering, updating and expanding ECO*_n™ and EMC*_n thence rendering, updating and expanding ECODI*_n rendering and successively adapting and expanding adaptable ECO*_n and EMC*_n purveying and procuring and pushing, pulling and streaming continuum*_n to, between and among relevant targeted and requesting networked repositories and associated touchscreens in, between and among individual*_n and individual group*_n (henceforth collectively known as interactives) in EMC*_n wherein CIPS instructing individual*_n and individual group*_n to rendering interactives in, between among said networked devices wherein interactives rendering and successively updating, improving and expanding said continuum*_n and said footprint*_n in said compositions and compilations in said storage means, in said interactives and footprint*_n in, between and among individual*_n and individual group*_n whereby interactives rendering and successively updating, improving and expanding individual*_n and individual group*_n in EMC*_n to rendering and successively editing, updating, adapting, improving and expanding ECODI*_n thence rendering and successively editing, updating, adapting, improving and expanding abiotic thinking and reasoning of ECO*_n thence rendering and successively editing, updating, adapting, improving and expanding individual*_n and individual group*_n being supported by commercial computer programs and software and Wi-Fi and hot spots to instant and rapid edge-computing and edge-exchanging exchange*_n of continuum*_n and footprint*_n, wireless and cellular technologies and derivatives and speech recognition and near-field technologies to rendering and successively editing, updating, adapting and expanding footprint*_n rendering and successively editing, updating, adapting and expanding individual*_n and individual group*_n in EMC*_n;
CIPS instructing said networked repositories to rendering and successively adapting and expanding adaptable Infinite edge-computing and edge-exchanging Oracle to rendering, improving and expanding adaptable abiotic thinking and reasoning to supplanting present and future random and fragmented thought processes and decisions to rendering decisions and related outcomes, occurences and sequelae, and said Infinite edge-computing and edge-exchanging Oracle to rendering and successively expanding and enhancing said network repositories-rendered abiotic, thinking and reasoning to managing, tracking and trending said continuum*_n of diagnoses of acute, subacute and chronic diseases and disorders and sequelae and related or relevant vital goods, products, services and resources in diverse industries in diverse phases and circumstances of healthcare, care-giving and long-term care associating with integral snippets*_n and relations of integral snippets*_n comprising relational data on demographics, medical, surgical and social histories and data of users of EMC*_n and identifications and FP addresses of said networked devices in, between and among said continuum*_n and digital footprint*_n in, between and among said networked repositories wherein CIPS instructing said networked repositories to rendering said compositions and compilations as interactive selectable and clickable relational data in integral snippets*_n and relations of integral snippets*_n in interactive touchscreens and interactive, selectable and clickable multi-dimensional universal network data-driven visualizations integral snippets*_n and relations of integral snippets*_n in EMC*_n (henceforth collectively known as adaptable and expanding ECODI*_n aka ECODI*_n); and
ECO*_n in, between and among individual*_n and individual group*_n using CIPS to instructing said networked repositories in individual*_n and individual group*_n to rendering instant-rendered inherent, new, adapable and unique or instantly flash or real-time inherent security digital token*_n handshaking between and among individual*_n and individual group*_n wherein said security digital token*_n to managing, tracking, trending and protecting said individual*_n and individual group*_n in EMC*_n.

12. The process according to claim 11 wherein said integral snippets*_n and relations of integral snippets*_n comprising relational data comprises said ECO*_n in, between and among individual*_n and individual group*_n to rendering and successively editing, updating, adapting, improving and expanding industry*_n-specific chronologically sequenced and sequentially time-stamped relevant specialties-, categories- and headings of diagnosis*_n-specific medical history*_n- and surgical*_n-specific differential diagnosis*_n, cause*_n, chief complaints, signs and symptoms, review of systems, physical examination, laboratory tests and results, related, asociated and relevant relational data on individual diagnoses and relational diagnoses and associated differential diagnoses related to continuum*_n on diagnoses of acute, subacute and chronic diseases and disorders and sequelae and related or relevant vital goods, products, services and resources in diverse industries in diverse phases and circumstances of healthcare care-giving and long-term care.

13. The process according to claim 11 wherein edge-computing and edge-exchanging mobile*_n-to-mobile*_n communication devices rendering and successively editing, updating, adapting, improving and expanding compositions and compilations of said integral snippets*_n and relations in, between and among said integral snippets*_n in said relational databases comprising ECO*_n rendering and successively editing, updating, adapting, improving and expanding continuum*_n including digital footprint*_n continuum*_n, between and among individual*_n and individual group*_n in EMC*_n, said relational databases comprises relational data of medical, surgical and social specialty-specific, diagnosis-specific, syndrome-specific and medical history specialties-, categories- and headings-specific snippets of relational data of networked repositories-digital id*_n, -timestamped, -temporally and -chronologically ordered relational sequences of present illneses, past medical history, past surgical history, vaccinations, general health, infectious diseases, sources of infections, accidents, injuries, repetitive strain injuries, hospitalizations, social history, substance abuse, recreation drug use, family history, travel history, occupations, avocations, asesments and plans, pharmaceuticals and medications, supplements, allergies, sideeffects and complications, procedures, treatments, therapies, outcomes, comments and feedbacks, associating with relational data in pharmaceuticals, intelligence, pharmaceuticals, financials, insurance, investments, estate plannings, actuarial services, community and social services, acute and chronic long-term, care-giving and end-of-life care, legal services, communication, real estate, travel and transportation, hotel and hospitality industries in said continuum*_n including digital footprint*_n continuum*_n in said relational databases in, between and among individual*_n and individual group*_n in EMC*_n.

14. The process according to claim 11 wherein ECO*_n rendering and successively editing, updating, adapting, improving and expanding said adaptable and expanding integral snippets*_n and relations in, between and among said integral. snippets*_n in said relational databases comprises ECO*_n rendering and successively editing, updating, adapting, improving and expandingrelational words, terms, phrases, graphics, pictures, videos, relational digital contents, phrases, sentences and any combinations thereof on said industries-, specialties-, categories- and headings-specific goods, products, services and resources and related financials and insurance products, services and resources in laypeople's languages and professionals' languages.

15. The process according to claim 11 wherein said interactives rendering and successively editing, updating, adapting, improving and expanding said adaptable and expanding continuum*_n including digital footprint*_n continuum*_n on diagnosis*_n of acute, subacute and chronic diseases and disorders and sequelae including metabolic syndrome, metabolism of fats, lipids, lipoproteins, proteins and carbohydrates, coronary atherosclerosis risks (CAR), cardiovascular diseases (CVD) and cerebrovascular diseases and related and associated diagnoses of diseases, disorders and sequelae and related or relevant vital goods, products, services and resources in diverse industries in diverse phases and circumstances of care-giving, long-term care and living.
